(19) United States
(12) Patent Application Publication
(10) Pub. No.: US 2023/0277464 A1
(43) Pub. Date: Sep. 7, 2023

(54) TABLET DOSAGE FORMS FOR LIPID-BASED DRUG DELIVERY SYSTEMS

(71) Applicant: ABITEC Corporation, Columbus, OH (US)

(72) Inventors: John Tillotson, Columbus, OH (US); Donald Kelemen, Columbus, OH (US)

(21) Appl. No.: 18/117,295

(22) Filed: Mar. 3, 2023

Related U.S. Application Data
(60) Provisional application No. 63/316,664, filed on Mar. 4, 2022.

(51) Int. Cl. A61K 9/20; A61K 31/192; A61K 31/05

(52) U.S. Cl. CPC A61K 9/2013; A61K 9/2068; A61K 9/2027; A61K 9/2059; A61K 9/2054; A61K 9/2009; A61K 9/2095; A61K 31/192; A61K 31/05

(57) ABSTRACT

Disclosed are tablet dosage forms and methods for formulating and delivering drugs via lipid-based drug delivery systems. An example tablet dosage form includes a plurality of granules, each granule comprising a pre-concentrate and a carbohydrate sorbent particle, where the pre-concentrate includes a drug having a log P of about −3 to about 10 and a lipid component The disclosed tablet dosage forms can be manufactured through high speed tableting methods with advantageous properties such as high drug release and low friability.

### CROSS-REFERENCE TO RELATED APPLICATIONS

This application claims priority to U.S. Provisional Patent Application No. 63/316,664 filed on Mar. 4, 2022, which is incorporated by reference herein in its entirety.

### TECHNICAL FIELD

This disclosure relates to tablet dosage forms that can be used for the formulation and delivery of drugs using lipid-based drug delivery systems.

### BACKGROUND

Lipid-based drug delivery systems, such as self-emulsifying drug delivery systems and single lipid drug delivery systems, are useful for in vivo delivery of drugs. Most lipid-based drug delivery systems are formulated as pre-concentrates which typically are incorporated into liquid or gel capsule dosage forms. A solid, directly compressible tablet dosage form for lipid-based drug delivery systems would be beneficial for broadening its application.

### SUMMARY

In one aspect, disclosed are tablet dosage forms including a plurality of granules, each granule including a pre-concentrate including a drug having a log P of about −3 to about 10 and a lipid component including at least one lipid, and a carbohydrate sorbent particle.

In another aspect, disclosed are tablet dosage forms including a plurality of granules, each granule including a pre-concentrate including a drug having a log P of about −3 to about 10, a lipid component including a combination of mono, di, and triglycerides, and a surfactant, and a carbohydrate sorbent particle.

In another aspect, disclosed are methods of making tablet dosage forms, the method including mixing a drug having a log P of about −3 to about 10 and a lipid component including at least one lipid to provide a pre-concentrate; adsorbing the pre-concentrate onto a plurality of carbohydrate sorbent particles and granulating to provide a plurality of granules; mixing the plurality of granules with a tableting diluent to provide a tableting mixture; and compressing the tablet mixture to provide a tablet dosage form.

### BRIEF DESCRIPTION OF THE DRAWINGS

FIG. 1 is an image showing an example pre-concentrate after 24 hours at room temperature.

FIG. 2 is an image showing the punch face of an upper punch of a tableting press used in an example method to provide an example tablet dosage form.

FIG. 3 is an image showing the punch face of an upper punch of a tableting press used in an example method to provide an example tablet dosage form.

FIG. 4 is an image showing the punch face of a lower punch of a tableting press used in an example method to provide an example tablet dosage form.

FIG. 5 is an image showing the punch face of a lower punch of a tableting press used in an example method to provide an example tablet dosage form.

FIG. 6 is a plot showing an example drug release from an example tablet dosage form.

### DETAILED DESCRIPTION

Among other things, disclosed herein is a tablet dosage form that can be used to formulate lipid-based drug delivery systems. The tablet dosage form includes a plurality of granules where each granule includes a pre-concentrate—that includes a drug, a lipid component, and an optional surfactant—and a carbohydrate sorbent particle. The unique tablet formulation can allow tablets to both advantageously carry lipid-based drug delivery systems and effectively release drugs from the tablet. In addition, the disclosed tablet formulation can allow the tablets to be produced by high-speed tableting techniques, which is beneficial for large-scale production.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the disclosed invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are explicitly contemplated in addition to 6 and 9, for the range 6.0-7.0, the number 6.0, 6.1, 6.2, 6.3, 6.4, 6.5, 6.6, 6.7, 6.8, 6.9, and 7.0 are explicitly contemplated, and for the range 1.5 to 2, the numbers 1.5, 1.6, 1.7, 1.8, 1.9, and 2 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “emulsion” as used herein refers to a system of two immiscible liquid phases. One of the two phases (the internal phase) is distributed as droplets/globules throughout the second phase (the external, or continuous phase). Emulsions include oil-in-water (o/w) emulsions, in which a less polar liquid commonly referred to as an oil is in the internal phase; and water-in-oil (w/o) emulsions, in which an aqueous or other relatively polar liquid is in the internal phase.

The term “glyceride” as used herein refers to lipophilic compounds comprising a glycerol molecule bonded to fatty acid groups. Monoglycerides are glycerol molecules bonded to a single fatty acid group; diglycerides are glycerol molecules bonded to two fatty acid groups; and triglycerides are glycerol molecules bonded to three fatty acid groups.

The term “fatty acid” as used herein refers to carboxylic acids with lipophilic chains comprising carbon and hydrogen atoms. Specific fatty acids can be identified by counting the number of carbon atoms and determining other chemical properties, such as the presence and location of double bonds between the carbon atoms, any branching of carbon atoms off the main lipophilic chain, and the presence of other atomic species in the chain. Fatty acids may be described as “saturated” (no double bonds between the carbon atoms), “monounsaturated” (one double bond between the carbon atoms), or “polyunsaturated” (more than one double bond between the carbon atoms). In some instances, the number of carbon atoms in a fatty acid is indicated by the prefix “C_{x-y}” or “Cₓ₋C_{y-}”, wherein x is the minimum and y is the maximum number of carbon atoms in the fatty acid. Thus, for example, “C₆₋₂₂ fatty acid” or “C₆-C₂₂ fatty acid” refers to a fatty acid containing from 6 to 22 carbon atoms.

The term “small molecule” refers to inorganic or organic chemical drugs having a molecular weight of less than 3,000 daltons.

The term “adsorb” includes both adsorption and absorption, meaning that it includes the processes of a first component, which can also be referred to as a “sorbent” (e.g., a carbohydrate sorbent particle) incorporating a second component (e.g., pre-concentrate) into the first component in any manner, including into its surface and bulk volume. Unless specifically indicated otherwise, the words “adsorb” and “absorb” are used interchangeably.

### 2. Tablet Dosage Forms

Provided herein are tablet dosage forms that can be used for lipid-based drug delivery systems. The tablet includes a plurality of granules, where each granule includes a pre-concentrate and a carbohydrate sorbent particle, and the pre-concentrate includes a drug, a lipid component, and an optional surfactant. The tablet may also include optional tablet excipients. Tablet dosage form and tablet are used interchangeably herein.

In some embodiments, the tablet dosage form includes a plurality of granules, each granule including a pre-concentrate that includes a drug having a log P of about −3 to about 10 and a lipid component including at least one lipid, and a carbohydrate sorbent particle; and an optional tablet excipient selected from the group consisting of a disintegrant, a diluent, a lubricant, and a combination thereof.

In some embodiments, the tablet dosage form includes a plurality of granules, each granule including a pre-concentrate that includes a drug having a log P of about −3 to about 10 and a lipid component including at least one lipid, and a carbohydrate sorbent particle; and an optional tablet excipient selected from the group consisting of a disintegrant, a diluent, a lubricant, an anti-adherent, and a combination thereof.

In some embodiments, the tablet dosage form includes a plurality of granules, each granule including a pre-concentrate that consists of a drug having a log P of about −3 to about 10, a lipid component including at least one lipid, and an optional surfactant, and a carbohydrate sorbent particle; and an optional tablet excipient selected from the group consisting of a disintegrant, a diluent, a lubricant, and a combination thereof.

In some embodiments, the tablet dosage form includes a plurality of granules, each granule consisting of a pre-concentrate that consists of a drug having a log P of about −3 to about 10, a lipid component including at least one lipid, and an optional surfactant, a carbohydrate sorbent particle, and an optional granule excipient selected from the group consisting of a disintegrant, a diluent, a binder, and a combination thereof, and an optional tablet excipient selected from the group consisting of a disintegrant, a diluent, a lubricant, and a combination thereof.

In some embodiments, the tablet dosage form consists of a plurality of granules, each granule consisting of a pre-concentrate that consists of a drug having a log P of about −3 to about 10, a lipid component including at least one lipid, and an optional surfactant, a carbohydrate sorbent particle, and an optional granule excipient selected from the group consisting of a disintegrant, a diluent, a binder, and a combination thereof, and an optional tablet excipient selected from the group consisting of a disintegrant, a diluent, a lubricant, and a combination thereof.

A. Granules

The tablet includes a plurality of granules. The granules are prepared by granulation of the pre-concentrate and the carbohydrate sorbent particle where the pre-concentrate can be adsorbed onto the carbohydrate sorbent particle. Accordingly, the granule can include the pre-concentrate and the carbohydrate sorbent particle. The granule can also include optional granule excipients, such as a suitable binder or a diluent (e.g., silicified microcrystalline cellulose).

The granules can include the pre-concentrate and carbohydrate sorbent particle in varying amounts, and as such, the tablet can also include the pre-concentrate and carbohydrate sorbent particle in varying amounts. For example, the pre-concentrate and the carbohydrate sorbent particle can be included in the tablet at a weight ratio of about 4:1 to about 1000:1 (particle:pre-concentrate), such as about 10:1 to about 1000:1, about 50:1 to about 500:1, about 7:1 to about 750:1, or about 10:1 to about 500:1.

The plurality of granules can be included in the tablet at varying amounts. For example, the tablet can include the plurality of granules at about 10% to about 80% by weight of the tablet, such as about 15% to about 75% by weight of the tablet, about 20% to about 80% by weight of the tablet, or about 15% to about 70% by weight of the tablet. In some embodiments, the tablet includes the plurality of granules at greater than 10% by weight of the tablet, greater than 15% by weight of the tablet, greater than 20% by weight of the tablet, greater than 25% by weight of the tablet, greater than 30% by weight of the tablet, greater than 35% by weight of the tablet, greater than 40% by weight of the tablet, greater than 45% by weight of the tablet, or greater than 50% by weight of the tablet. In some embodiments, the tablet includes the plurality of granules at less than 80% by weight of the tablet, less than 75% by weight of the tablet, less than 70% by weight of the tablet, less than 65% by weight of the tablet, less than 60% by weight of the tablet, less than 55% by weight of the tablet, less than 50% by weight of the tablet, less than 45% by weight of the tablet, or less than 40% by weight of the tablet.

In some embodiments, the granule consists of the pre-concentrate, the carbohydrate sorbent particle, and an optional granule excipient.
i. Pre-concentrates

The pre-concentrate is a lipid-based drug delivery system. The pre-concentrate includes a drug, a lipid component having at least one lipid, and optionally a surfactant. The pre-concentrate can enhance dissolution of the drug into aqueous fluids and/or absorption into the body, e.g., compared to the drug administered without the pre-concentrate. Examples of lipid-based drug delivery systems include self-emulsifying drug delivery systems (SEDDS), single lipid systems, polar lipid systems, and charged lipid systems. In some embodiments, the pre-concentrate is a SEDDS or a single lipid system. In some embodiments, the pre-concentrate is a SEDDS.

The pre-concentrate can be adsorbed on the carbohydrate sorbent particle and can be included in the tablet as granules. While the pre-concentrate and granule thereof can be in a solid form as part of the tablet, the pre-concentrate can be maintained as an oil system for digestion by organisms, or in the case of SEDDS, in a state that can be readily converted to an emulsion in vivo. For example, when the pre-concentrate contacts an aqueous environment, such as body fluid or gastrointestinal fluid, the pre-concentrate can form an emulsion (e.g., o/w emulsion) that includes the drug, the lipid component, and the optional surfactant.

The pre-concentrate can form varying sized emulsions. For example, the emulsion may have a particle size of about 0.01 μm to about 1 μm, such as about 0.05 μm to about 0.90 μm, about 0.1 μm to about 1 μm, about 0.08 μm to about 0.8 μm, or about 0.05 μm to about 1 μm. In some embodiments, the emulsion has a particle size of greater than 0.01 μm, greater than 0.05 μm, greater than 0.1 μm, greater than 0.2 μm, greater than 0.3 μm, greater than 0.4 μm, or greater than 0.5 μm. In some embodiments, the emulsion has a particle size of less than 1 μm, less than 0.9 μm, less than 0.8 μm, less than 0.7 μm, less than 0.6 μm, less than 0.5 μm, or less than 0.4 μm. The particle size of the emulsion may be measured by particle size analysis techniques that include, but are not limited to, laser diffraction, dynamic light scattering, and image analysis (e.g., using a microscopic method such as light microscopy or scanning electron microscopy).

In some embodiments, the pre-concentrate consists of a drug having a log P of about −3 to about 10, a lipid component including at least one lipid, and an optional surfactant, wherein the lipid is selected from the group consisting of pegylated ester, ethoxylated oil, hydrogenated vegetable oil, fatty acids, glycerol esters of fatty acids, propylene glycol esters of fatty acids, polyglycerol esters of fatty acids, polyethylene glycol, macrogolglycerides, polysorbates, polar and/or amphiphilic lipids, and a combination thereof.
a. Drug

The drug can be any drug that would benefit from administration as part of the disclosed pre-concentrate and tablet thereof. The term “drug” is conventional, referring to a compound having beneficial prophylactic and/or therapeutic properties when administered to a subject. Drugs having varying solubilities in water can benefit from the present disclosure. For example, a drug that exhibits appreciable water solubility can benefit from the increased bioavailability made possible by this disclosure if the formulation can reduce the size of the dose needed for therapeutic efficacy or increase the rate of drug absorption in cases where a rapid onset of the drug's effectiveness is desired. In addition, drugs having a low water solubility can benefit as described above, as well as be aided in their transport in aqueous environments. Accordingly, the drug can be hydrophobic, hydrophilic, or amphiphilic.

The drug can have a Log P of about −3 to about 10, such as about −2.5 to about 9, about −2 to about 8.5, about −1 to about 10, or about 0 to about 9. In some embodiments, the drug has a Log P of greater than −3, greater than −2, greater than −1, greater than 0, greater than 1, greater than 2, greater than 3, or greater than 4. In some embodiments, the drug has a Log P of less than 10, less than 9.5, less than 9, less than 8.5, less than 8, less than 7.5, less than 7, less than 6.5, or less than 6. Log P is the log(concentration of a compound in octanol/concentration of the compound in water). Log P=0 implies equal affinity for organic and aqueous phase; Log P<1 implies affinity towards aqueous phase; and Log P>1 implies affinity towards organic phase. Log P can be estimated, e.g., using Crippen's fragmentation method in the software ChemDraw.

In some embodiments, the drug is hydrophobic. For example, in some embodiments, the drug has a log P of about 1 to about 10, such as about 2 to about 10 or about 3 to about 10.

The granule may also include other therapeutic molecules, such as therapeutic biological molecules. Examples of other therapeutic molecules include, but are not limited to, peptides, proteins, oligonucleotides, and small molecule drugs. In some embodiments, the granule further includes a peptide, a protein, an oligonucleotide, a small molecule drug, or a combination thereof.

The drug can be included in the tablet in varying amounts. For example, the tablet can include the drug at about 0.010% to about 30% by weight of the tablet, such as about 0.05% to about 25% by weight of the tablet, about 0.10% to about 20% by weight of the tablet, about 1% to about 30% by weight of the tablet, about 0.01% to about 20% by weight of the tablet, or about 0.01% to about 10% by weight of the tablet. In some embodiments, the tablet includes the drug at greater than 0.010% by weight of the tablet, greater than 0.1% by weight of the tablet, greater than 0.5% by weight of the tablet, greater than 1% by weight of the tablet, greater than 5% by weight of the tablet, greater than 10% by weight of the tablet, or greater than 15% by weight of the tablet. In some embodiments, the tablet includes the drug at less than 30% by weight of the tablet, less than 25% by weight of the tablet, less than 20% by weight of the tablet, less than 15% by weight of the tablet, less than 10% by weight of the tablet, less than 5% by weight of the tablet, or less than 1% by weight of the tablet.
b. Lipid Components

The pre-concentrate includes a lipid component. The lipid component can include at least one lipid. For example, in some embodiments, the lipid component includes a single lipid, while in other embodiments, the lipid component includes more than one lipid. Accordingly, in some embodiments, the lipid component includes a plurality or mixture of lipids.

Any lipid suitable for forming the pre-concentrate can be used in the lipid component. Example lipids include, but are not limited to, pegylated ester, ethoxylated oil, hydrogenated vegetable oil, fatty acids, glycerol esters of fatty acids, propylene glycol esters of fatty acids, polyglycerol esters of fatty acids, polyethylene glycol, macrogolglycerides, polysorbates, polar and/or amphiphilic lipids, and combinations thereof.

The lipid component can include short chain (e.g., less than 6 carbon atoms), medium chain (e.g., 6-12 carbon atoms), and long chain (e.g., greater than 12 carbon atoms) fatty acids and esters thereof. The fatty acid or ester thereof can be saturated or unsaturated. In addition, the fatty acid ester can be a mono, a di, or a tri-fatty acid ester. In some embodiments, the lipid component includes a C₆-C₂₂ fatty acid or ester thereof. In some embodiments, the lipid component includes a C₆-C₁₆ fatty acid or ester thereof. In some embodiments, the lipid component includes a C₆-C₁₂ fatty acid or ester thereof.

In some embodiments, the lipid component includes an individual type of short chain, medium chain, or long chain fatty acid or ester thereof. In some embodiments, the lipid component includes a combination of short chain, medium chain, and/or long chain fatty acids or esters thereof. In some embodiments, the lipid component includes an individual type of C₆-C₂₂ fatty acid or ester thereof. In some embodiments, the lipid component includes a combination of different C₆-C₂₂ fatty acids or esters thereof.

As mentioned above, the lipid component can include glycerol esters of fatty acids. For example, the lipid component can include a monoglyceride, a diglyceride, a triglyceride, or a combination thereof. In some embodiments, the lipid component includes a mono, a di, a triglyceride, or a combination thereof having a C₆-C₂₂ fatty acid. In some embodiments, the lipid component includes a mono, a di, a triglyceride, or a combination thereof having a C₆-C₁₆ fatty acid. In some embodiments, the lipid component includes a mono, a di, a triglyceride, or a combination thereof having a C₆-C₁₂ fatty acid. In addition, the lipid component can include polyglycerols, which include multiple units of glycerol atomically connected with glycerides. For example, polyglycerol esters can have 3 to 10 units of glycerol with fatty acids (e.g., mono, di, or tri) esterified to them.

Examples of commercially available lipids include, but are not limited to, various grades of the following commercial products: CAPTEX®; MIGLYOL®; and CAPMUL® (available in different grades, e.g., Capmul MCM).

The lipid component can be included in the tablet at varying amounts. For example, the tablet can include the lipid component at about 0.1% to about 35% by weight of the tablet, such as about 0.2% to about 30% by weight of the tablet, about 0.5% to about 25% by weight of the tablet, about 0.1% to about 20% by weight of the tablet, or about 0.5% to about 15% by weight of the tablet. In some embodiments, the tablet includes the lipid component at greater than 0.10% by weight of the tablet, greater than 0.50% by weight of the tablet, greater than 10% by weight of the tablet, greater than 5% by weight of the tablet, greater than 10% by weight of the tablet, or greater than 15% by weight of the tablet. In some embodiments, the tablet includes the lipid component at less than 35% by weight of the tablet, less than 30% by weight of the tablet, less than 25% by weight of the tablet, less than 20% by weight of the tablet, less than 15% by weight of the tablet, or less than 10% by weight of the tablet.
c. Surfactants

The pre-concentrate may also include a surfactant. The pre-concentrate can include a single surfactant or a plurality of surfactants. The surfactant may be anionic, amphiphilic, non-ionic, or cationic. In addition, the surfactant may have a hydrophile-lipophile balance (HLB) value of greater than or equal to 8, such as greater than or equal to 8.5, greater than or equal to 9, greater than or equal to 9.5, or greater than or equal to 10. In some embodiments, the surfactant has a HLB value of less than or equal to 17, such as less than or equal to 16.5, less than or equal to 16, less than or equal to 15.5, or less than or equal to 15. In some embodiments, the surfactant has a HLB value of about 7 to about 17, such as about 7.5 to about 16, about 8 to about 15, or about 9 to about 16.

Any suitable pharmaceutically acceptable surfactant may be used in the disclosed pre-concentrates. Examples of surfactants include, but are not limited to, pegylated esters, macrogolglycerides, vitamin E TPGS, proteins (such as casein), lecithin, cholesterol, propylene glycol, polyglycerol esters of fatty acids, acid and ester ethoxylates of fatty acids, sorbitan esters of fatty acids, alcohol ethoxylates, polyoxyethylene or polyoxypropylene copolymers, phospholipids, polyoxyethylene sorbitan fatty acid derivatives (such as polysorbates), castor oil or hydrogenated castor oil ethoxylates, such as ethoxylated oil or polyethoxylated oil, anionic surfactants, such as sodium lauryl sulphate or sodium oleate, and alkylphenol surfactants.

While there may be overlap between components that can be used as part of the lipid component and that can be used as the surfactant, embodiments that have both components have different components for each. For example, a pre-concentrate having a surfactant can include polyglycerol esters of fatty acids as the at least one lipid, but that same embodiment does not include polyglycerol esters of fatty acids as both the surfactant and the at least one lipid.

In some embodiments, the surfactant includes pegylated ester, macrogolglycerides, polysorbates, vitamin E TPGS, ethoxylated oil, polyethoxylated oil, or a combination thereof. In some embodiments, the surfactant includes pegylated ester, macrogolglycerides, polysorbates, vitamin E TPGS, ethoxylated oil, or polyethoxylated oil. In some embodiments, the surfactant includes ethoxylated oil or polyethoxylated oil.
ii. Carbohydrate Sorbent Particles

The carbohydrate sorbent particle can be any particle that is made from a carbohydrate or polysaccharide source and that has the capacity to adsorb the pre-concentrate and components thereof, such as oils, lipids, drugs, and surfactants. Adsorption of the pre-concentrate on the carbohydrate sorbent particle can provide the granules thereof as a free-flowing powder (e.g., via a granulation unit), which can be tableted.

The carbohydrate sorbent particle provides advantageous benefits to the disclosed tablet dosage form. For example, the carbohydrate sorbent particle has a beneficial lipid carrying capacity combined with beneficial release of drug from the tablet. The carbohydrate sorbent particle can have a lipid carrying capacity of about 0.10% to about 355% by weight of the carbohydrate sorbent particle, such as about 0.2% to about 30%, about 0.5% to about 20%, about 0.10% to about 25%, or about 0.50% to about 35% by weight of the carbohydrate sorbent particle. In some embodiments, the carbohydrate sorbent particle has a lipid carrying capacity of greater than 0.10% by weight of the carbohydrate sorbent particle, greater than 0.5% by weight of the carbohydrate sorbent particle, greater than 1% by weight of the carbohydrate sorbent particle, greater than 5% by weight of the carbohydrate sorbent particle, greater than 10% by weight of the carbohydrate sorbent particle, or greater than 15% by weight of the carbohydrate sorbent particle. In some embodiments, the carbohydrate sorbent particle has a lipid carrying capacity of less than 35% by weight of the carbohydrate sorbent particle, less than 30% by weight of the carbohydrate sorbent particle, less than 25% by weight of the carbohydrate sorbent particle, less than 20% by weight of the carbohydrate sorbent particle, less than 15% by weight of the carbohydrate sorbent particle, or less than 10% by weight of the carbohydrate sorbent particle.

Examples of suitable carbohydrate sorbent particles include, but are not limited to, maltodextrin and silicified MCC. In some embodiments, the carbohydrate sorbent particle includes maltodextrin, silicified MCC, or a combination thereof. In some embodiments, the carbohydrate sorbent particle includes maltodextrin or silicified MCC. In some embodiments, the carbohydrate sorbent particle includes maltodextrin. In some embodiments, the carbohydrate sorbent particle does not include a silicate-based adsorbent carrier, such as silica (e.g., Aerosil® and Aeroperl®), magnesium aluminometasilicate (e.g., Neusilin®), and the like.

The carbohydrate sorbent particle can have a varying particle size. For example, the carbohydrate sorbent particle can have a diameter of about 1 μm to about 1000 μm. The particle size of the carbohydrate sorbent particle may be measured by particle size analysis techniques that include, but are not limited to, laser diffraction, dynamic light scattering, sieve separation analysis and image analysis (e.g., using a microscopic method such as light microscopy or scanning electron microscopy).

As mentioned above, the carbohydrate sorbent particle is from a carbohydrate or polysaccharide source. For example, the carbohydrate sorbent particle can be greater than 85%, greater than 90%, greater than 93%, greater than 95%, greater than 97%, or greater than 99% carbohydrate or polysaccharide based on the weight of the carbohydrate sorbent particle. In some embodiments, the carbohydrate sorbent particle is less than 100%, less than 99%, less than 98%, less than 97%, less than 96%, or less than 95% carbohydrate or polysaccharide based on the weight of the carbohydrate sorbent particle. In some embodiments, the carbohydrate sorbent particle is about 85% to about 100% carbohydrate or polysaccharide based on the weight of the carbohydrate sorbent particle, such as about 85% to about 99% carbohydrate or polysaccharide based on the weight of the carbohydrate sorbent particle, about 90% to about 100% carbohydrate or polysaccharide based on the weight of the carbohydrate sorbent particle, or about 90% to about 99% carbohydrate or polysaccharide based on the weight of the carbohydrate sorbent particle.

The carbohydrate sorbent particle can be included in the tablet at varying amounts. For example, the tablet can include the carbohydrate sorbent particle at about 10% to about 60% by weight of the tablet, such as about 15% to about 55% by weight of the tablet, about 20% to about 50% by weight of the tablet, about 20% to about 60% by weight of the tablet, or about 10% to about 50% by weight of the tablet. In some embodiments, the tablet includes the carbohydrate sorbent particle at greater than 10% by weight of the tablet, greater than 15% by weight of the tablet, greater than 20% by weight of the tablet, greater than 25% by weight of the tablet, greater than 30% by weight of the tablet, greater than 35% by weight of the tablet, or greater than 40% by weight of the tablet. In some embodiments, the tablet includes the carbohydrate sorbent particle at less than 60% by weight of the tablet, less than 55% by weight of the tablet, less than 50% by weight of the tablet, less than 45% by weight of the tablet, less than 40% by weight of the tablet, less than 35% by weight of the tablet, or less than 30% by weight of the tablet.
iii. Granule Excipients

The granules can further include a granule excipient. Granule excipients include, but are not limited to, diluents, disintegrants, and binders. Examples of diluents include, but are not limited to, mannitol, maltitol, maltodextrin, micro-crystalline cellulose, silicified micro-crystalline celluloses, dextrates, polyols, lactose, sugars, polysaccharides, starches, glucose, gum, xanthine, calcium salt, silica, silicate, and combinations thereof. Examples of disintegrants include, but are not limited to, sodium starch glycolate, crospovidone, crosslinked sodium carboxymethyl cellulose, starch, and combinations thereof. And, examples of binders include, but are not limited to, hydroxypropyl methyl cellulose, hydroxyethyl cellulose, hydroxypropyl cellulose, carboxymethyl cellulose, xanthan gum, carrageenan, acacia gum, polyvinylpyrrolidone, chitosan, methyl cellulose, poly-vinyl acetate, povidone, ethyl acrylate, methyl methacrylate, methacrylic acid ester, and combinations thereof.

In some embodiments, each granule includes a diluent, a disintegrant, and a binder. In some embodiments, the granule includes a diluent selected from the group consisting of mannitol, maltitol, maltodextrin, micro-crystalline cellulose, and silicified micro-crystalline cellulose; a disintegrant selected from the group consisting of crospovidone and crosslinked sodium carboxymethyl cellulose; and a binder selected from the group consisting of hydroxypropyl methyl cellulose, hydroxyethyl cellulose, hydroxypropyl cellulose, carboxymethyl cellulose, povidone, and polyvinylpyrrolidone.

Granule excipients can be included in the tablet in varying amounts. For example, the tablet can include the granule diluent at about 1% to about 60% by weight of the tablet, such as about 5% to about 60% by weight of the tablet, about 10% to about 60% by weight of the tablet, about 1% to about 50% by weight of the tablet, or about 10% to about 40% by weight of the tablet. In some embodiments, the tablet includes the granule diluent at greater than 1% by weight of the tablet, greater than 5% by weight of the tablet, greater than 10% by weight of the tablet, or greater than 15% by weight of the tablet. In some embodiments, the tablet includes the granule diluent at less than 60% by weight of the tablet, less than 55% by weight of the tablet, less than 50% by weight of the tablet, or less than 45% by weight of the tablet.

The tablet can include the granule disintegrant at about 0% to about 10% by weight of the tablet, such as about 0.5% to about 10% by weight of the tablet, about 0% to about 5% by weight of the tablet, about 0.5% to about 8% by weight of the tablet, about 0.5% to about 5% by weight of the tablet, or about 1% to about 10% by weight of the tablet. In some embodiments, the tablet includes the granule disintegrant at greater than 0% by weight of the tablet, greater than 0.5% by weight of the tablet, greater than 10% by weight of the tablet, or greater than 2% by weight of the tablet. In some embodiments, the tablet includes the granule disintegrant at less than 10% by weight of the tablet, less than 9% by weight of the tablet, less than 7% by weight of the tablet, or less than 5% by weight of the tablet.

And, the tablet can include the granule binder at about 0.5% to about 10% by weight of the tablet, such as about 1% to about 10% by weight of the tablet, about 0.5% to about 5% by weight of the tablet, about 5% to about 10% by weight of the tablet, about 0.8% to about 8% by weight of the tablet, or about 1% to about 7% by weight of the tablet. In some embodiments, the tablet includes the granule binder at greater than 0.5% by weight of the tablet, greater than 1% by weight of the tablet, greater than 1.5% by weight of the tablet, or greater than 2% by weight of the tablet. In some embodiments, the tablet includes the granule binder at less than 10% by weight of the tablet, less than 9% by weight of the tablet, less than 7% by weight of the tablet, or less than 5% by weight of the tablet.

In some embodiments, the tablet includes the granule excipient (e.g., diluents, disintegrants, binders, or combinations thereof) at about 0% to about 80% by weight of the tablet, such as about 0.5% to about 80% by weight of the tablet, about 1% to about 80% by weight of the tablet, about 1% to about 70% by weight of the tablet, about 0.5% to about 50% by weight of the tablet, about 5% to about 75% by weight of the tablet, about 0.5% to about 60% by weight of the tablet, about 20% to about 80% by weight of the tablet, or about 40% to about 80% by weight of the tablet. In some embodiments, the tablet includes the granule excipient at greater than 0% by weight of the tablet, greater than 5% by weight of the tablet, greater than 10% by weight of the tablet, or greater than 20% by weight of the tablet. In some embodiments, the tablet includes the granule excipient at less than 80% by weight of the tablet, less than 70% by weight of the tablet, less than 60% by weight of the tablet, or less than 50% by weight of the tablet.

B. Tablet Excipients

The tablet dosage form can further include tablet excipients. Tablet excipients include, but are not limited to, diluents, disintegrants (e.g., extra-granular disintegrants), anti-adherents, and lubricants. The description above regarding the diluent and the disintegrant for the granules can also be applied to the tablet excipients. With respect to the lubricant, examples include, but are not limited to, sodium stearyl fumarate, magnesium stearate, calcium stearate, hydrogenated vegetable oil, stearic acid, metal stearate, talc, wax, polyethylene glycol, boric acid, sodium benzoate, sodium acetate, sodium oleate, sodium lauryl sulphate, magnesium lauryl sulphate, and combinations thereof. An example anti-adherent includes, but is not limited to, silicon dioxide.

In some embodiments, the tablet excipient includes a diluent, a disintegrant, and a lubricant. In some embodiments, the tablet excipient includes a diluent, a disintegrant, a lubricant, and an anti-adherent. In some embodiments, the tablet excipient includes a diluent selected from the group consisting of dextrates, polyols, lactose, sugars, polysaccharides, starches, celluloses, glucose, and gum; a disintegrant selected from the group consisting of crospovidone, sodium starch glycolate, and crosslinked sodium carboxymethyl cellulose; and a lubricant selected from the group consisting of sodium stearyl fumarate, magnesium stearate, calcium stearate, hydrogenated vegetable oil, stearic acid, and metal stearate.

Tableting excipients can be included in the tablet in varying amounts. For example, the tablet can include the diluent at about 10% to about 90% by weight of the tablet, such as about 10% to about 80% by weight of the tablet, about 10% to about 50% by weight of the tablet, about 30% to about 90% by weight of the tablet, or about 50% to about 90% by weight of the tablet. In some embodiments, the tablet includes the diluent at greater than 10% by weight of the tablet, greater than 20% by weight of the tablet, greater than 30% by weight of the tablet, or greater than 40% by weight of the tablet. In some embodiments, the tablet includes the diluent at less than 90% by weight of the tablet, less than 80% by weight of the tablet, less than 70% by weight of the tablet, or less than 60% by weight of the tablet.

The tablet can include the disintegrant at about 0.5% to about 10% by weight of the tablet, such as about 0.5% to about 8% by weight of the tablet, about 0.5% to about 5% by weight of the tablet, about 2% to about 8% by weight of the tablet, or about 5% to about 10% by weight of the tablet. In some embodiments, the tablet includes the disintegrant at greater than 0.5% by weight of the tablet, greater than 1% by weight of the tablet, greater than 1.5% by weight of the tablet, or greater than 2% by weight of the tablet. In some embodiments, the tablet includes the disintegrant at less than 10% by weight of the tablet, less than 9.5% by weight of the tablet, less than 9% by weight of the tablet, or less than 8.5% by weight of the tablet.

The tablet can include the lubricant at about 0.1% to about 5% by weight of the tablet, such as about 0.5% to about 5% by weight of the tablet, about 1% to about 5% by weight of the tablet, about 0.1% to about 4% by weight of the tablet, or about 0.10% to about 2% by weight of the tablet. In some embodiments, the tablet includes the lubricant at greater than 0.10% by weight of the tablet, greater than 0.5% by weight of the tablet, greater than 0.9% by weight of the tablet, or greater than 1% by weight of the tablet. In some embodiments, the tablet includes the lubricant at less than 5% by weight of the tablet, less than 4.5% by weight of the tablet, less than 4% by weight of the tablet, or less than 3.5% by weight of the tablet.

And, the tablet can include the anti-adherent at about 0.1% to about 5% by weight of the tablet, such as about 0.5% to about 5% by weight of the tablet, about 1% to about 5% by weight of the tablet, about 0.1% to about 4% by weight of the tablet, or about 0.1% to about 2% by weight of the tablet. In some embodiments, the tablet includes the anti-adherent at greater than 0.10% by weight of the tablet, greater than 0.5% by weight of the tablet, greater than 0.9% by weight of the tablet, or greater than 1% by weight of the tablet. In some embodiments, the tablet includes the anti-adherent at less than 5% by weight of the tablet, less than 4.5% by weight of the tablet, less than 4% by weight of the tablet, or less than 3.5% by weight of the tablet.

In some embodiments, the tablet includes the tablet excipient (e.g., diluents, disintegrants, anti-adherents, lubricants, or combinations thereof) at about 0.1% to about 90% by weight of the tablet, such as about 0.1% to about 80% by weight of the tablet, about 0.1% to about 70% by weight of the tablet, about 1% to about 75% by weight of the tablet, about 0.10% to about 50% by weight of the tablet, about 0.10% to about 40% by weight of the tablet, about 10% to about 90% by weight of the tablet, about 20% to about 90% by weight of the tablet, or about 50% to about 90% by weight of the tablet. In some embodiments, the tablet includes the tablet excipient at greater than 0.10% by weight of the tablet, greater than 5% by weight of the tablet, greater than 10% by weight of the tablet, or greater than 30% by weight of the tablet. In some embodiments, the tablet includes the tablet excipient at less than 90% by weight of the tablet, less than 80% by weight of the tablet, less than 70% by weight of the tablet, or less than 60% by weight of the tablet.

C. Tablet Properties

The components of the tablet dosage form can afford the tablet advantageous properties. For example, the tablet dosage form can have useful friability characteristics. The tablet may have a friability of less than 1.0% after friability testing according to USP 43-NF 38 section <1216> Tablet friability, such as less than 0.9%, less than 0.8%, less than 0.7%, less than 0.6%, or less than 0.5% according to USP 43-NF 38 section <1216> Tablet friability. In some embodiments, the tablet has a friability of greater than 0.1% after friability testing according to USP 43-NF 38 section <1216> Tablet friability, such as greater than 0.2%, greater than 0.3%, greater than 0.4%, greater than 0.5%, or greater than 0.6% according to USP 43-NF 38 section <1216> Tablet friability. In some embodiments, the tablet has a friability of about 0.1% to about 1.0% after friability testing according to USP 43-NF 38 section <1216> Tablet friability, such as about 0.2% to about 0.9% or about 0.5% to about 1% after friability testing according to USP 43-NF 38 section <1216> Tablet friability. The tablet dosage form can also be produced with high uniformity. For example, the tablet may pass the USP/NF Uniformity of Dosage Units <905> criteria. In addition, the tablet dosage form can have little to no tack. Or in other words, the tablet can be tack-free. Overall, these properties allow the disclosed tablets to be manufactured, e.g., by high-speed rotary tableting equipment.

The tablet dosage form can have advantageous release properties. For example, the tablet can release the drug at greater than 50% by weight as measured by the tablet being exposed to a mixing solution (also referred to as a dissolution media) of 0.1 N HCl at about 50 rpm and at about 37° C., such as greater than 55%, greater than 60%, greater than 65%, greater than 70%, greater than 75%, greater than 80%, or greater than 90% as measured by the tablet being exposed to a dissolution media of 0.1 N HCl at about 50 rpm and at about 37° C. In some embodiments, the tablet releases the drug at less than 99% by weight as measured by the tablet being exposed to a dissolution media of 0.1 N HCl at about 50 rpm and at about 37° C., such as less than 95%, less than 90%, less than 85%, less than 80%, less than 75%, less than 70%, or less than 65% as measured by the tablet being exposed to a dissolution media of 0.1 N HCl at about 50 rpm and at about 37° C. In some embodiments, the tablet releases the drug at about 50% to about 99% by weight as measured by the tablet being exposed to a dissolution media of 0.1 N HCl at about 50 rpm and at about 37° C., such as about 55% to about 95% or about 65% to about 99% by weight as measured by the tablet being exposed to a dissolution media of 0.1 N HCl at about 50 rpm and at about 37° C. In some embodiments, the tablet allows for complete release of the drug as measured by the tablet being exposed to a dissolution media of 0.1 N HCl at about 50 rpm and at about 37° C.

The tablet dosage form may have a useful hardness. In some embodiments, the tablet has a hardness of about 2 kP to about 10 kP, such as about 3 kP to about 9 kP, about 4 kP to about 8 kP, about 5 kP to about 9 kP, or about 6 kP to about 8 kP. In some embodiments, the tablet has a hardness of greater than 2 kP, greater than 3 kP, or greater than 4 kP. In some embodiments, the tablet has a hardness of less than 10 kP, less than 9 kP, or less than 8 kP.

The tablet dosage form may have a varying thickness, shape, or both depending, in part, on the methods of making (e.g., punch configuration) and the components used.

In some embodiments, the tablet has a friability of less than 1.0% after friability testing according to USP 43-NF 38 section <1216> Tablet friability; passes USP/NF Uniformity of Dosage Units <905> criteria; releases the drug at greater than 50% by weight as measured by the tablet being exposed to a dissolution media of 0.1 N HCl at about 50 rpm and at about 37° C.; or a combination thereof.

### 3. Methods

The present disclosure also provides methods of making tablet dosage forms. The method can include mixing a drug having a log P of about −3 to about 10 and a lipid component including at least one lipid to provide a pre-concentrate. In some embodiments, the lipid component includes a single lipid, such as a short chain, medium chain, or long chain fatty acid or ester thereof. In other embodiments, the lipid component includes a plurality or combination of lipids, such as a combination of short chain, medium chain, or long chain fatty acids or esters thereof. In some embodiments, the lipid component includes a combination of mono, di, and triglycerides. The mixing step can also include a surfactant being mixed with the drug and the lipid component.

Mixing of the drug, lipid component, and optional surfactant can be done in any suitable manner that can provide the pre-concentrate. Mixing can be carried out for about 1 minute to about 60 minutes. In some embodiments, the mixing is carried out for greater than 1 minute, greater than 5 minutes, or greater than 10 minutes. In some embodiments, the mixing is carried out for less than 60 minutes, less than 45 minutes, or less than 30 minutes. Mixing can be carried out by any suitable mixing apparatus, such as a magnetic stirrer. In some embodiments, heat can be added to provide the pre-concentrate. In some embodiments, the lipid component and optional surfactant can be mixed first, which can then be followed by addition of the drug.

The method can include adsorbing the pre-concentrate onto a plurality of carbohydrate sorbent particles and granulating to provide a plurality of granules. Adsorbing the pre-concentrate can be carried out via a variety of techniques known within the art, such as spraying the pre-concentrate. After the pre-concentrate has been adsorbed onto the plurality of carbohydrate sorbent particles, they can then be granulated. Granulation can be carried out with suitable methods known within the art, such as a high-shear granulator or a fluid-bed granulator. Granulation can also include addition of optional granule excipients to the pre-concentrate adsorbed carbohydrate sorbent particles. In some embodiments, a disintegrant, a diluent, or a combination thereof is included in granulating. The method can also include contacting the plurality of carbohydrate sorbent particles with a binder solution. The binder solution can be added (e.g., by spraying) during the granulation process. The binder solution can include binders as listed above and a solvent. The solvent can include water, alcohols, organic solvents, or a combination thereof. The provided plurality of granules can be dried, screened through a separating mesh, or a combination thereof.

The method can further include compressing the plurality of granules or tablet mixture thereof to provide a tablet dosage form. Prior to tableting, the method can include mixing the plurality of granules with an optional tableting excipient(s). In some embodiments, the plurality of granules are mixed with a disintegrant, a diluent, a lubricant, an anti-adherent, or a combination thereof. The compressing step can include using a tableting press, such as a rotary tablet press or other suitable presses. Due to the advantageous properties of the disclosed tablet formulation, the produced tablets can avoid significantly sticking to the tableting press and tableting tool. For example, in some embodiments, the tablet does not stick or does not significantly stick to the tableting press, tableting tooling, or both. This is beneficial for high-speed tableting press methods. Accordingly, compressing can be done via high speed tableting methods. For example, compressing can include having dwell times of about 1 msec to about 30 msec. In some embodiments, compressing includes a dwell time of greater than 1 msec, greater than 5 msec, or greater than 10 msec. In some embodiments, compressing includes a dwell time of less than 30 msec, less than 25 msec, or less than 20 msec. In some embodiments, compressing includes a compression force of about 5 kN to about 40 kN. In some embodiments, compressing includes a compression force of greater than 5 kN, greater than 10 kN, or greater than 15 kN. In some embodiments, compressing includes a compression force of less than 40 kN, less than 35 kN, or less than 30 kN. In some embodiments, compressing includes an RPM of about 10 to about 100. In some embodiments, compressing includes an RPM of greater than 10, greater than 25, or greater than 50. In some embodiments, compressing includes an RPM of less than 100, less than 75, or less than 50.

The description above regarding the tablet dosage forms (and properties thereof), granules, pre-concentrates, drugs, lipid components, surfactants, carbohydrate sorbent particles, granule excipients, and tablet excipients can also be applied to the methods disclosed herein.

### 4. EXAMPLES

### Example 1

### SEDDS Formulation

SEDDS ibuprofen tablets were successfully manufactured at industry dwell times. With heating, about 15.0% of Ibuprofen was dissolved in the pre-concentrate components and remained dissolved at room temperature for at least 24 hours. The manufactured tablets were of low hardness, but had very low friability. No sticking of tableting ingredients was observed on the upper or lower punches during tableting. Slight oily residue was noticed, but this is expected when tableting oils.

Pre-concentrate: The pre-concentrate was prepared on a w/w basis according to the formulation in Table 1. 1000 g of pre-concentrate was prepared.

TABLE 1
Pre-concentrate formulation

| | | Weight |
|---|---|---|
| Ingredient | % | (g) |
| ibuprofen | 15 | 150 |
| triglyceride of medium chain fatty acid | 29.1 | 291 |
| mono and diglycerides of medium chain | 29.1 | 291 |
| fatty acids | | |
| polyethoxylated castor oil | 26.8 | 268 |

The medium chain fatty acid esters and oil were weighed into a 2000 mL glass beaker. Subsequently, a magnetic stirrer was added to the beaker. The medium chain fatty acid esters and oil were blended to uniformity for 5 minutes with the magnetic stirrer set at 5. Subsequently, the ibuprofen was added and levigated into the pre-concentrate with a spatula for wetting, and then blended in with the magnetic stirrer on high and heat set at 5 until visually dissolved. Visual dissolution occurred in about 15 minutes with the hot plate surface temperature being about 190.0-195.4° C. and the pre-concentrate surface temperature being about 30.1-33.1° C. The pre-concentrate was observed 24 hours later to ensure that the ibuprofen was still dissolved.

Binder solution: Binder solution manufacturing was prepared by weighing 120 g of polyvinylpyrrolidone into a 1000 mL stainless steel beaker and blending it into 400 g of 99% isopropyl alcohol weighed into the stainless-steel beaker with a Lightnin labmaster overhead stirrer set at 200 rpm and outfitted with a large asp blade. During blending the 1000 mL stainless-steel beaker was covered with plastic to prevent evaporation of the alcohol. Blending was maintained until all polyvinylpyrrolidone was dissolved (about 2 hours). The binder solution was set aside for subsequent use.

SEDDS granulation: The SEDDS granulation ingredients were weighed out as indicated in Table 2.

TABLE 2
SEDDS granulation formulation

| | | Weight |
|---|---|---|
| Ingredient | % | (g) |
| SEDDS pre-concentrate | 10 | 100 |
| maltodextrin | 74.5 | 745 |
| cross-linked sodium carboxymethyl cellulose | 1.5 | 15 |
| silicified microcrystalline cellulose | 8 | 80 |
| polyvinylpyrrolidone | 6 | 60 |
| Total | 100 | 1000 |

The maltodextrin was placed in the bowl of a Diosna lab-scale high-shear granulator set at a blade speed of 200 rpm and a chopper speed of 50 rpm. An airless sprayer under 60 psi and outfitted with a 650067 nozzle was employed to spray the SEDDS pre-concentrate onto the maltodextrin. After completion of the SEDDS pre-concentrate spray, the high-shear granulator blending continued for an additional 3 minutes. Subsequently, the granulator was stopped, and the silicified microcrystalline cellulose and cross-linked sodium carboxymethyl cellulose was added to the granulator. These were blended in the high-shear granulator with the maltodextrin and SEDDS pre-concentrate combination for approximately 1 minute at 200 rpm blade speed and 50 rpm chopper speed. Subsequently, 260 g of granulating solution (60 g of polyvinylpyrrolidone), was sprayed onto the components in the high-shear granulator employing the same airless sprayer, 60 psi, and same nozzle. Once the granulation solution was sprayed on completely, the high-shear granulator continued to mix for 1 minute, and then, the granulator was stopped. The contents of the granulator were emptied onto a stainless-steel pan and evenly spread out. The stainless-steel pan with granulation was then placed in a tray-drier set at 55° C. and allowed to dry for 24 hours. After 24 hours, the dried granulation was removed from the oven and screened through a #16-mesh and collected for further processing.

SEDDS tablet: SEDDS tableting ingredients were weighed out as indicated in Table 3.

TABLE 3
SEDDS tableting formulation

| | | Weight |
|---|---|---|
| Ingredient | % | (g) |
| SEDDS Granulation | 77 | 385 |
| combination of monosaccharides and | 20 | 100 |
| polysaccharides | | |
| cross-linked sodium carboxymethyl cellulose | 1 | 5 |
| fumed silica | 0.5 | 2.5 |
| sodium stearyl fumarate | 1.5 | 7.5 |
| Total | 100 | 500 |

The combination of monosaccharides and polysaccharides, cross-linked sodium carboxymethyl cellulose, fumed silica, and sodium stearyl fumarate were passed through a #20-mesh. All ingredients with the exception of sodium stearyl fumarate were added to a 3-qt V-blender and blended for 5 minutes at 25 rpm. Subsequently, the sodium stearyl fumarate was added to the V-blender and blended for 2 minutes at 25 rpm. The resultant blend was tableted into 1000 mg tablets on a 16-station Manesty rotary tablet press outfitted with 0.625″ FFBE Type “B” tooling operated at 50 rpm with a compression force of 5400 lbs. Tooling was inspected for residue after each tableting run.

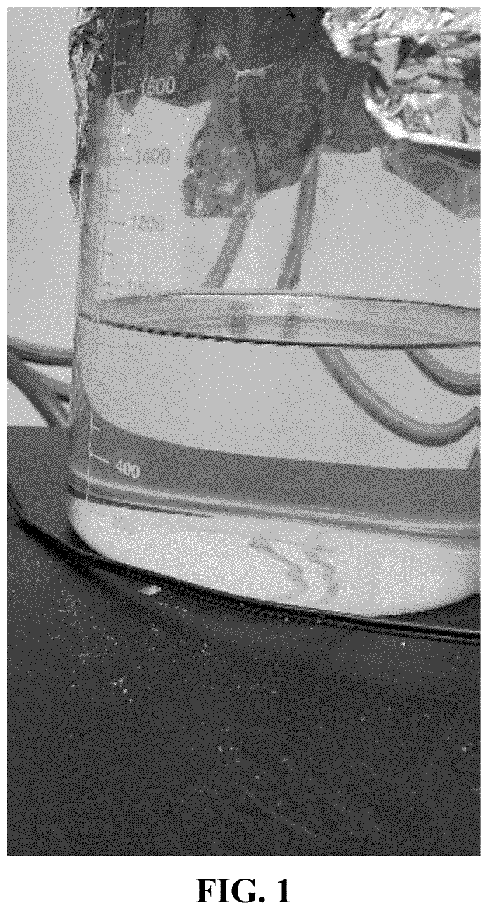

Results: After heating, the ibuprofen was completely dissolved in the SEDDS pre-concentrate components and remained dissolved after 24 hours (FIG. 1).

SEDDS ibuprofen tablets were successfully manufactured without creating sticking or picking on the punches. Table 4 displays the tableting characteristics for the ibuprofen SEDDS tablets.

TABLE 4
Tablet characteristics of SEDDS tablets

| Tablet | Weight (mg) | Thickness (mm) | Hardness (kP) |
|---|---|---|---|
| 1 | 1026 | 4.40 | 7.5 |
| 2 | 1058 | 4.59 | 7.2 |
| 3 | 1035 | 4.45 | 7.4 |
| 4 | 1036 | 4.43 | 8.0 |
| 5 | 1050 | 4.52 | 7.1 |
| | 1041.0 (+/−) 12.8 | 4.48 (+/−) 0.08 | 7.4 (+/−) 0.4 |

Friability=0.1%
Compression force=5431 lbs
Ejection force=19 lbs
RPM=51
Dwell time 10 msec

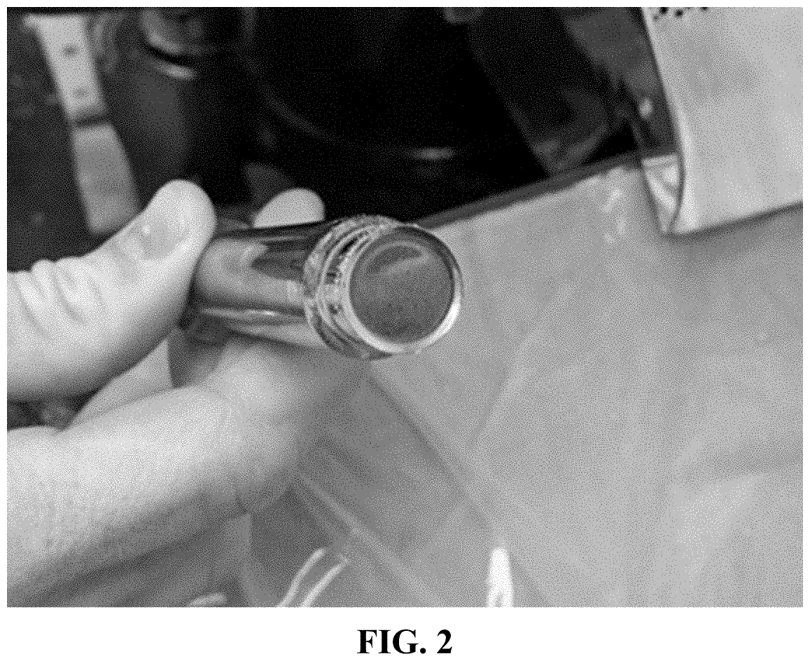

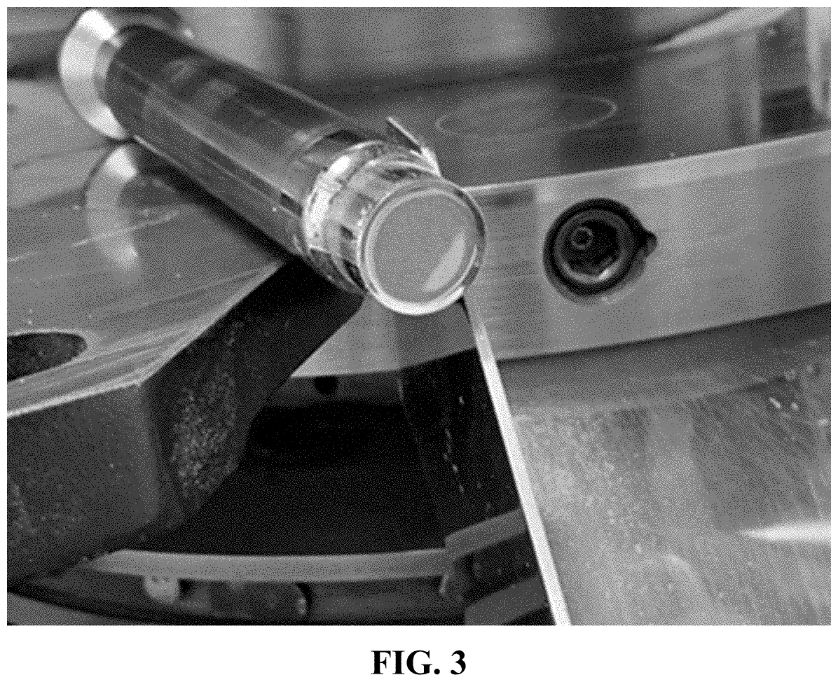

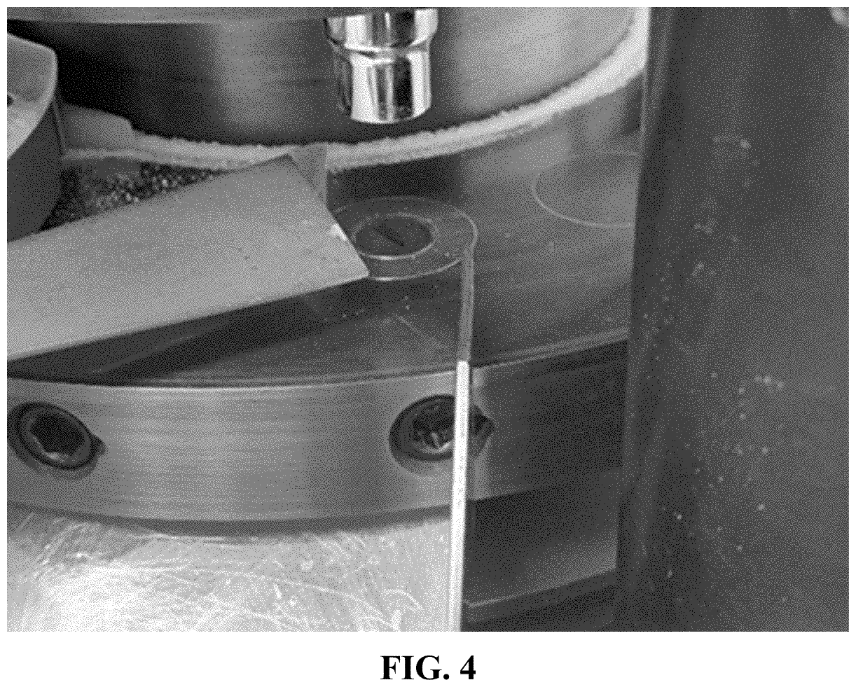

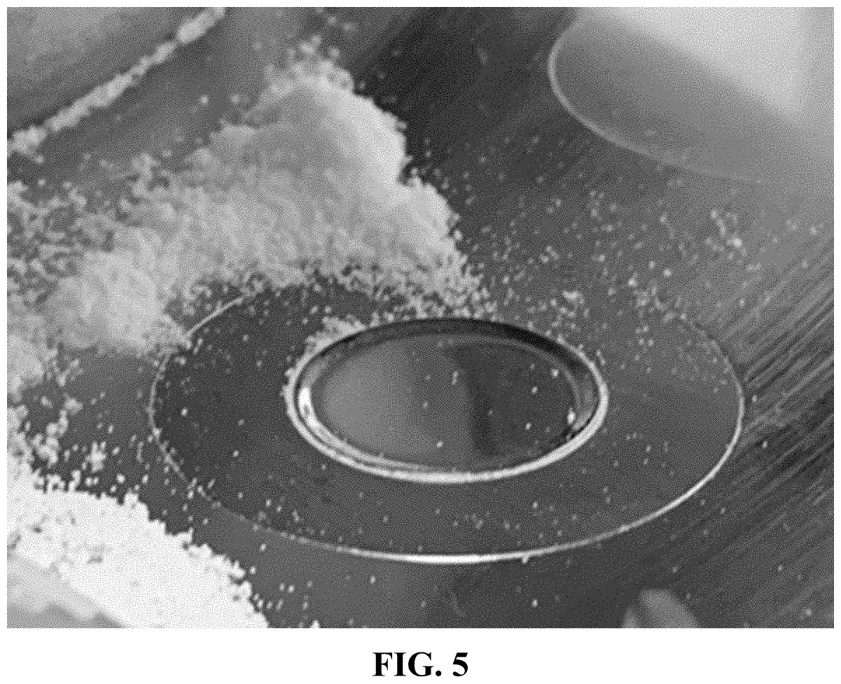

Punches did not exhibit sticking or picking over the tableting run (FIG. 2, FIG. 3, FIG. 4, and FIG. 5). Some oil residue was noticed, but this is expected when tableting oil containing excipients.

Tablets were tested for content uniformity as indicated under the Uniformity of Dosage Units in the USP (Table 5).

TABLE 5
Uniformity of Dosage Units

| Tablet Number | Ibuprofen content (mg) | % Label Claim |
|---|---|---|
| 1 | 10.76 | 93.1 |
| 2 | 10.79 | 93.4 |
| 3 | 11.06 | 95.8 |
| 4 | 10.56 | 91.5 |
| 5 | 10.65 | 92.2 |
| 6 | 10.68 | 92.5 |
| 7 | 10.64 | 92.1 |
| 8 | 10.48 | 90.7 |
| 9 | 10.31 | 89.2 |
| 10 | 10.76 | 93.2 |

Average percent of label claim=92.4%
Standard deviation=1.7
L1 acceptance value=10.3<15→pass

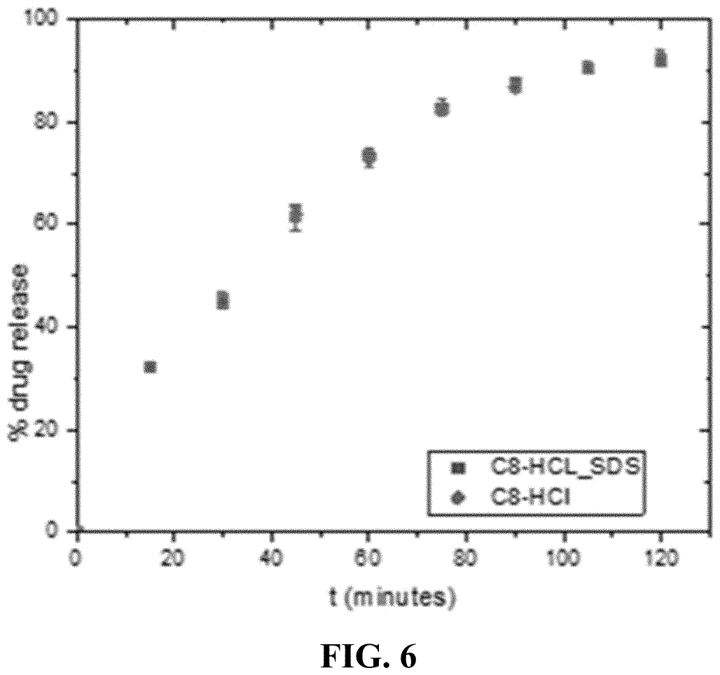

Ibuprofen release was determined by employing a USP apparatus 2 at 50 rpm in 450 mL of 0.1 NHCL at 37.5° C. (with and without SLS (also referred to as SDS) (0.5%)). Results are shown in FIG. 6 and Tables 6 and 7 below.

TABLE 6
Release without SLS

| | mg lbup | | % average | |
|---|---|---|---|---|
| C8-HCL | average jar | | Ibuprofen | |
| Time/min | 2 + jar 3 | sd | release | sd |
| 0 | 0 | 0 | 0 | 0 |
| 30 | 5.30 | 0.15 | 45.69 | 1.30 |
| 45 | 7.19 | 0.17 | 62.02 | 1.50 |
| 60 | 8.44 | 0.19 | 72.77 | 1.63 |
| 75 | 9.56 | 0.05 | 82.39 | 0.42 |
| 90 | 10.06 | 0.05 | 86.70 | 0.43 |
| 105 | 10.51 | 0.14 | 90.61 | 1.17 |
| 120 | 10.73 | 0.19 | 92.52 | 1.65 |
| 1440 | 10.96 | 0.20 | 93.25 | 1.71 |

TABLE 7
Release with SLS

| | mg lbup | | average | |
|---|---|---|---|---|
| C8-HCL-SLS | average | | ibuprofen | |
| time/min | jar2 + jar3 | sd | release | sd |
| 0 | 0 | 0 | 0 | 0 |
| 20 | 3.72 | 0.09 | 32.09 | 0.80 |
| 30 | 5.20 | 0.01 | 44.86 | 0.08 |
| 45 | 7.13 | 0.29 | 61.45 | 2.51 |
| 60 | 8.49 | 0.21 | 73.23 | 1.84 |
| 75 | 9.60 | 0.18 | 82.73 | 1.57 |
| 90 | 10.16 | 0.08 | 87.58 | 0.69 |
| 105 | 10.49 | 0.06 | 90.45 | 0.49 |
| 120 | 10.65 | 0.04 | 91.78 | 0.38 |
| 1440 | 10.25 | 0.10 | 88.40 | 0.90 |

### Example 2

### Single Lipid Formulation

The pre-concentrate is prepared on a w/w basis according to a formulation that includes CBD isolate as the drug and a triglyceride of a medium chain fatty acid as the single lipid.

Pre-concentrate: 800 g of triglyceride of medium chain fatty acid is added to 2000 mL glass beaker. A magnetic stirrer is added to the beaker and set to agitate at 5. Subsequently, 200 g of CBD isolate is weighed and added to the beaker. The CBD isolate is levigated with a spatula into the pre-concentrate for wetting. Blending continues by magnetic stirrer set at 5 until the CBD Isolate is visually dissolved.

Binder solution: Binder solution manufacturing is prepared by weighing 120 g of polyvinylpyrrolidone into a 1000 mL stainless steel beaker and blending it into 400 g of 99% isopropyl alcohol weighed into the stainless-steel beaker with a Lightnin labmaster overhead stirrer set at 200 rpm and outfitted with a large asp blade. During blending the 1000 mL stainless-steel beaker is covered with plastic to prevent evaporation of the alcohol. Blending is maintained until all polyvinylpyrrolidone is dissolved (typically about 2 hours). The binder solution is set aside for subsequent use.

SEDDS granulation: The SEDDS granulation ingredients are weighed out as indicated in Table 8.

TABLE 8
SEDDS granulation formulation

| | | Weight |
|---|---|---|
| Ingredient | % | (g) |
| SEDDS pre-concentrate | 10 | 100 |
| maltodextrin | 74.5 | 745 |
| cross-linked sodium carboxymethyl cellulose | 1.5 | 15 |
| silicified microcrystalline cellulose | 8 | 80 |
| polyvinylpyrrolidone | 6 | 60 |
| Total | 100 | 1000 |

The maltodextrin is placed in the bowl of a Diosna lab-scale high-shear granulator set at a blade speed of 200 rpm and a chopper speed of 50 rpm. An airless sprayer under 60 psi and outfitted with a 650067 nozzle is employed to spray the SEDDS pre-concentrate onto the maltodextrin. After completion of the SEDDS pre-concentrate spray, the high-shear granulator blending continues for an additional 3 minutes. Subsequently, the granulator is stopped, and the silicified microcrystalline cellulose and cross-linked sodium carboxymethyl cellulose are added to the granulator. These are blended in the high-shear granulator with the maltodextrin and SEDDS pre-concentrate combination for approximately 1 minute at 200 rpm blade speed and 50 rpm chopper speed. Subsequently, 260 g of granulating solution (60 g of polyvinylpyrrolidone), is sprayed onto the components in the high-shear granulator employing the same airless sprayer, 60 psi, and same nozzle. Once the granulation solution is sprayed on completely, the high-shear granulator continues to mix for 1 minute, and then, the granulator is stopped. The contents of the granulator are emptied onto a stainless-steel pan and evenly spread out. The stainless-steel pan with granulation is then placed in a tray-drier set at 55° C. and allowed to dry for 24 hours. After 24 hours, the dried granulation is removed from the oven and screened through a #16-mesh and collected for further processing.

SEDDS tablet: SEDDS tableting ingredients are weighed out as indicated in Table 9.

TABLE 9
SEDDS tableting formulation

| | | Weight |
|---|---|---|
| Ingredient | % | (g) |
| SEDDS Granulation | 77 | 385 |
| combination of monosaccharides and | 20 | 100 |
| polysaccharides | | |
| cross-linked sodium carboxymethyl cellulose | 1 | 5 |
| fumed silica | 0.5 | 2.5 |
| sodium stearyl fumarate | 1.5 | 7.5 |
| Total | 100 | 500 |

The combination of monosaccharides and polysaccharides, cross-linked sodium carboxymethyl cellulose, fumed silica, and sodium stearyl fumarate is passed through a #20-mesh. All ingredients with the exception of sodium stearyl fumarate are added to a 3-qt V-blender and blended for 5 minutes at 25 rpm. Subsequently, the sodium stearyl fumarate is added to the V-blender and blended for 2 minutes at 25 rpm. The resultant blend is tableted into 1000 mg tablets on a 16-station Manesty rotary tablet press outfitted with 0.625″ FFBE Type “B” tooling operated at 50 rpm with a compression force of 5400 lbs.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A tablet dosage form comprising: a plurality of granules, each granule comprising a pre-concentrate including a drug having a log P of about −3 to about 10 and a lipid component including at least one lipid, and a carbohydrate sorbent particle.

Clause 2. A tablet dosage form comprising: a plurality of granules, each granule comprising a pre-concentrate including a drug having a log P of about −3 to about 10, a lipid component including a combination of mono, di, and triglycerides, and a surfactant, and a carbohydrate sorbent particle.

Clause 3. The tablet dosage form of clause 1 or clause 2, wherein the pre-concentrate and the carbohydrate sorbent particle are included in the tablet at a weight ratio of about 4:1 to about 1000:1 (particle:pre-concentrate).

Clause 4. The tablet dosage form of any one of clauses 1-3, wherein the tablet includes the drug at about 0.01% to about 30% by weight of the tablet.

Clause 5. The tablet dosage form of any one of clauses 1-4, wherein the tablet includes the lipid component at about 0.10% to about 355% by weight of the tablet.

Clause 6. The tablet dosage form of any one of clauses 1-5, wherein the tablet includes the carbohydrate sorbent particle at about 10% to about 60% by weight of the tablet.

Clause 7. The tablet dosage form of any one of clauses 1-6, wherein the tablet includes the plurality of granules at about 10% to about 80% by weight of the tablet.

Clause 8. The tablet dosage form of any one of clauses 1-7, wherein the carbohydrate sorbent particle has a lipid carrying capacity of about 0.10% to about 35% by weight of the carbohydrate sorbent particle.

Clause 9. The tablet dosage form of any one of clauses 1-8, wherein the carbohydrate sorbent particle comprises maltodextrin, silicified micro-crystalline cellulose, or a combination thereof.

Clause 10. The tablet dosage form of any one of clauses 1-9, wherein the lipid component comprises pegylated ester, ethoxylated oil, hydrogenated vegetable oil, fatty acid, glycerol ester of a fatty acid, propylene glycol esters of fatty acids, polyglycerol ester of a fatty acid, polyethylene glycol, macrogolglycerides, polysorbates, polar and/or amphiphilic lipids, or a combination thereof.

Clause 11. The tablet dosage form of any one of clauses 1-10, wherein the lipid component includes a C₆-C₂₂ fatty acid or ester thereof.

Clause 12. The tablet dosage form of any one of clauses 1-11, wherein the surfactant comprises pegylated ester, macrogolglycerides, polysorbates, vitamin E TPGS, ethoxylated oil, polyethoxylated oil, or a combination thereof.

Clause 13. The tablet dosage form of any one of clauses 1-12, wherein the pre-concentrate is a self-emulsifying drug delivery system or a single lipid system.

Clause 14. The tablet dosage form of any one of clauses 1-13, wherein the pre-concentrate is adsorbed onto the carbohydrate sorbent particle.

Clause 15. The tablet dosage form of any one of clauses 1-14, wherein the pre-concentrate forms an emulsion with an average particle size of about 0.01 μm to about 1 μm when present in an aqueous environment.

Clause 16. The tablet dosage form of any one of clauses 1-15, wherein each granule further comprises a peptide, a protein, an oligonucleotide, a small molecule drug, or a combination thereof.

Clause 17. The tablet dosage form of any one of clauses 1-16, wherein each granule further comprises a diluent, a disintegrant, a binder, or a combination thereof.

Clause 18. The tablet dosage form of any one of clauses 1-17, further comprising a tablet excipient.

Clause 19. The tablet dosage form of clause 17, wherein the binder comprises hydroxypropyl methyl cellulose, hydroxyethyl cellulose, hydroxypropyl cellulose, carboxymethyl cellulose, xanthan gum, carrageenan, acacia gum, polyvinylpyrrolidone, povidone, chitosan, methyl cellulose, poly-vinyl acetate, povidone, ethyl acrylate, methyl methacrylate, methacrylic acid ester, or a combination thereof.

Clause 20. The tablet dosage form of clause 17 or clause 18, wherein the diluent comprises mannitol, maltitol, maltodextrin, micro-crystalline cellulose, silicified micro-crystalline celluloses, dextrate, polyol, lactose, sugar, polysaccharide, starch, glucose, gum, xanthine, calcium salt, silica, silicate, or a combination thereof.

Clause 21. The tablet dosage form of clause 17 or clause 18, wherein the disintegrant comprises sodium starch glycolate, crospovidone, crosslinked sodium carboxymethyl cellulose, starch, or a combination thereof.

Clause 22. The tablet dosage form of clause 18, wherein the tablet excipient comprises a lubricant selected from the group consisting of sodium stearyl fumarate, magnesium stearate, calcium stearate, hydrogenated vegetable oil, stearic acid, metal stearate, talc, wax, polyethylene glycol, boric acid, sodium benzoate, sodium acetate, sodium oleate, sodium lauryl sulphate, magnesium lauryl sulphate, and a combination thereof.

Clause 23. The tablet dosage form of any one of clauses 1-22, wherein the tablet has a friability of less than 1.0% after friability testing according to USP 43-NF 38 section <1216> Tablet friability.

Clause 24. The tablet dosage form of any one of clauses 1-23, wherein the tablet passes USP/NF Uniformity of Dosage Units <905> criteria.

Clause 25. The tablet dosage form of any one of clauses 1-24, wherein the tablet releases the drug at greater than 50% by weight as measured by the tablet being exposed to a dissolution media of 0.1 N HCl at about 50 rpm and at about 37° C.

Clause 26. A method of making a tablet dosage form, the method comprising: mixing a drug having a log P of about −3 to about 10 and a lipid component including at least one lipid to provide a pre-concentrate; adsorbing the pre-concentrate onto a plurality of carbohydrate sorbent particles and granulating to provide a plurality of granules; mixing the plurality of granules with a tableting excipient to provide a tablet mixture; and compressing the tablet mixture to provide a tablet dosage form.

Clause 27. The method of clause 26, wherein the lipid component includes a combination of mono, di, and triglycerides.

Clause 28. The method of clause 26 or clause 27, wherein a surfactant is mixed with the drug and the lipid component.

Clause 29. The method of any one of clauses 26-28, comprising contacting the carbohydrate sorbent particles with a binder solution.

Clause 30. The method of any one of clauses 26-29, wherein compressing includes having a dwell time of about 1 msec to about 30 msec.

Clause 31. The method of any one of clauses 26-30, wherein the tablet is tack-free.

Clause 32. The method of any one of clauses 26-31, wherein compressing includes a tableting press and a tableting tooling and the tablet does not significantly adhere to the tableting press, tableting tooling, or both during or after compressing.

Clause 33. The method of any one of clauses 26-32, wherein the tablet has a friability of less than 1.0% after friability testing according to USP 43-NF 38 section <1216> Tablet friability; passes USP/NF Uniformity of Dosage Units <905> criteria; releases the drug at greater than 50% by weight as measured by the tablet being exposed to a dissolution media of 0.1 N HCl at about 50 rpm and at about 37° C.; or a combination thereof.

What is claimed is:

1. A tablet dosage form comprising:
a plurality of granules, each granule comprising
a pre-concentrate including a drug having a log P of about −3 to about 10 and a lipid component including at least one lipid, and
a carbohydrate sorbent particle.

2. A tablet dosage form comprising:
a plurality of granules, each granule comprising
a pre-concentrate including a drug having a log P of about −3 to about 10, a lipid component including a combination of mono, di, and triglycerides, and a surfactant, and
a carbohydrate sorbent particle.

3. The tablet dosage form of claim 1, wherein the pre-concentrate and the carbohydrate sorbent particle are included in the tablet at a weight ratio of about 4:1 to about 1000:1 (particle:pre-concentrate).

4. The tablet dosage form of claim 1, wherein the tablet includes the drug at about 0.01% to about 30% by weight of the tablet.

5. The tablet dosage form of claim 1, wherein the tablet includes the lipid component at about 0.10% to about 35% by weight of the tablet.

6. The tablet dosage form of claim 1, wherein the tablet includes the carbohydrate sorbent particle at about 10% to about 60% by weight of the tablet.

7. The tablet dosage form of claim 1, wherein the tablet includes the plurality of granules at about 10% to about 80% by weight of the tablet.

8. The tablet dosage form of claim 1, wherein the carbohydrate sorbent particle has a lipid carrying capacity of about 0.10% to about 35% by weight of the carbohydrate sorbent particle.

9. The tablet dosage form of claim 1, wherein the carbohydrate sorbent particle comprises maltodextrin, silicified micro-crystalline cellulose, or a combination thereof.

10. The tablet dosage form of claim 1, wherein the lipid component comprises pegylated ester, ethoxylated oil, hydrogenated vegetable oil, fatty acid, glycerol ester of a fatty acid, propylene glycol esters of fatty acids, polyglycerol ester of a fatty acid, polyethylene glycol, macrogolglycerides, polysorbates, polar and/or amphiphilic lipids, or a combination thereof.

11. The tablet dosage form of claim 1, wherein the lipid component includes a C₆-C₂₂ fatty acid or ester thereof.

12. The tablet dosage form of claim 2, wherein the surfactant comprises pegylated ester, macrogolglycerides, polysorbates, vitamin E TPGS, ethoxylated oil, polyethoxylated oil, or a combination thereof.

13. The tablet dosage form of claim 1, wherein the pre-concentrate is a self-emulsifying drug delivery system or a single lipid system.

14. The tablet dosage form of claim 1, wherein the pre-concentrate is adsorbed onto the carbohydrate sorbent particle.

15. The tablet dosage form of claim 1, wherein the pre-concentrate forms an emulsion with an average particle size of about 0.01 μm to about 1 μm when present in an aqueous environment.

16. The tablet dosage form of claim 1, wherein each granule further comprises a peptide, a protein, an oligonucleotide, a small molecule drug, or a combination thereof.

17. The tablet dosage form of claim 1, wherein each granule further comprises a diluent, a disintegrant, a binder, or a combination thereof.

18. The tablet dosage form of claim 1, further comprising a tablet excipient.

19. The tablet dosage form of claim 17, wherein the binder comprises hydroxypropyl methyl cellulose, hydroxyethyl cellulose, hydroxypropyl cellulose, carboxymethyl cellulose, xanthan gum, carrageenan, acacia gum, polyvinylpyrrolidone, povidone, chitosan, methyl cellulose, poly-vinyl acetate, povidone, ethyl acrylate, methyl methacrylate, methacrylic acid ester, or a combination thereof.

20. The tablet dosage form of claim 17, wherein the diluent comprises mannitol, maltitol, maltodextrin, micro-crystalline cellulose, silicified micro-crystalline cellulose, dextrate, polyol, lactose, sugar, polysaccharide, starch, glucose, gum, xanthine, calcium salt, silica, silicate, or a combination thereof.

21. The tablet dosage form of claim 17, wherein the disintegrant comprises sodium starch glycolate, crospovidone, crosslinked sodium carboxymethyl cellulose, starch, or a combination thereof.

22. The tablet dosage form of claim 18, wherein the tablet excipient comprises a lubricant selected from the group consisting of sodium stearyl fumarate, magnesium stearate, calcium stearate, hydrogenated vegetable oil, stearic acid, metal stearate, talc, wax, polyethylene glycol, boric acid, sodium benzoate, sodium acetate, sodium oleate, sodium lauryl sulphate, magnesium lauryl sulphate, and a combination thereof.

23. The tablet dosage form of claim 1, wherein the tablet has a friability of less than 1.0% after friability testing according to USP 43-NF 38 section <1216> Tablet friability.

24. The tablet dosage form of claim 1, wherein the tablet passes USP/NF Uniformity of Dosage Units <905> criteria.

25. The tablet dosage form of claim 1, wherein the tablet releases the drug at greater than 50% by weight as measured by the tablet being exposed to a dissolution media of 0.1 N HCl at about 50 rpm and at about 37° C.

26. A method of making a tablet dosage form, the method comprising:
mixing a drug having a log P of about −3 to about 10 and a lipid component including at least one lipid to provide a pre-concentrate;
adsorbing the pre-concentrate onto a plurality of carbohydrate sorbent particles and granulating to provide a plurality of granules;
mixing the plurality of granules with a tableting excipient to provide a tablet mixture; and
compressing the tablet mixture to provide a tablet dosage form.

27. The method of claim 26, wherein the lipid component includes a combination of mono, di, and triglycerides.

28. The method of claim 26, wherein a surfactant is mixed with the drug and the lipid component.

29. The method of claim 26, comprising contacting the carbohydrate sorbent particles with a binder solution.

30. The method of claim 26, wherein compressing includes having a dwell time of about 1 msec to about 30 msec.

31. The method of claim 26, wherein the tablet is tack-free.

32. The method of claim 26, wherein compressing includes a tableting press and a tableting tooling and the tablet does not significantly adhere to the tableting press, tableting tooling, or both during or after compressing.

33. The method of claim 26, wherein the tablet has a friability of less than 1.0% after friability testing according to USP 43-NF 38 section <1216> Tablet friability; passes USP/NF Uniformity of Dosage Units <905> criteria; releases the drug at greater than 50% by weight as measured by the tablet being exposed to a dissolution media of 0.1 N HCl at about 50 rpm and at about 37° C.; or a combination thereof.
